(19) United States
(12) Patent Application Publication
(10) Pub. No.: US 2025/0044313 A1
(43) Pub. Date: Feb. 6, 2025

(54) APPARATUS AND PROCESS FOR PERFORMING LATERAL FLOW TESTS

(71) Applicant: RANDOX LABORATORIES LTD., Crumlin (GB)

(72) Inventors: Ivan MCCONNELL, Crumlin (GB); Lewis MCKINSTRY, Crumlin (GB); Peter FITZGERALD, Crumlin (GB)

(73) Assignee: RANDOX LABORATORIES LTD., Crumlin (GB)

(21) Appl. No.: 18/717,649

(22) PCT Filed: Dec. 9, 2022
(86) PCT No.: PCT/GB2022/053148

§ 371 Date: Jun. 7, 2024

(30) Foreign Application Priority Data
Dec. 10, 2021 (GB) 2117887.6

(51) Int. Cl. G01N 35/02; G01N 33/543; G01N 35/00; G01N 35/04

(52) U.S. Cl. CPC G01N 35/025; G01N 33/54388; G01N 2035/00455; G01N 2035/0441

(57) ABSTRACT

An apparatus for assaying one or more lateral flow test devices. The apparatus comprises at least one support device for supporting one or more lateral flow test devices in use and at least one sample receptacle configured to receive, in use, a respective liquid sample to be dispensed onto a corresponding lateral flow test device. The apparatus further comprises a dispensing apparatus configured to dispense a known volume of the received liquid sample from the sample receptacle onto the corresponding lateral flow test device supported by the support device.

### FIELD OF THE INVENTION

The invention relates to an apparatus, system and method for assaying lateral flow test devices, for example of the kind suitable for detecting a SARS-COV-2 antigen.

### BACKGROUND TO THE INVENTION

A lateral flow test is a simple procedure for rapidly detecting the presence of a specific analyte such as a virus in a sample of biological material. In a lateral flow test, a liquid sample is deposited onto a strip of porous material (e.g. paper or microstructured polymer) that carries reactive molecules configured to produce a visual result indicative of the presence or absence of the analyte of interest. The test device is typically left to develop for a predetermined time period after which the visual result is read by the operator conducting the test. The visual result may for example take the form of a coloured test line which only appears when the analyte is present in the liquid sample.

Lateral flow tests have traditionally found application as a way of conducting fast, on-demand tests in clinical and laboratory settings. Recently, however, these tests have been widely adopted in response to the COVID-19 pandemic for mass testing of individuals for infection with SARS-COV-2. Favoured for their speed and simplicity of use, they are now often deployed at sites such as airports and event venues for testing the large numbers of individuals present, where they are typically performed by non-specialist personnel such as venue staff or indeed by the individuals being tested as a means of self-testing. Lateral flow tests for detecting a SARS-COV-2 antigen are typically conducted by collecting a nasal and/or throat swab, dipping the swab into a buffer solution in order to form a liquid sample and then dispensing, by hand, a predetermined number of drops of the liquid sample onto the sample zone of a lateral flow test device that carries the porous material and associated chemical agents needed to assay the sample.

While it is in principle an effective way of conducting high volumes of tests, this new application of lateral flow tests to mass testing in non-clinical settings suffers from a number of drawbacks. Since the tests are usually performed by non-specialist personnel, there is usually significant inconsistency in the preparation of the samples, their dispensation onto the test devices and the interpretation of the visual results both between operators and between individual tests. These issues are exacerbated by variations in environmental conditions such as temperature and lighting at these non-clinical sites and by the increased potential for contamination by debris and cross-contamination between tests that arises where large numbers of tests are performed in environments of this kind. There is hence a need for a way of conducting mass, in situ testing that overcomes these limitations.

### SUMMARY OF THE INVENTION

A first aspect of the invention provides an apparatus for assaying one or more lateral flow test devices, the apparatus comprising: at least one support device for supporting one or more lateral flow test devices in use; at least one sample receptacle configured to receive, in use, a respective liquid sample to be dispensed onto a corresponding lateral flow test device; and a dispensing apparatus configured to dispense a known volume of the received liquid sample from the sample receptacle onto the corresponding lateral flow test device supported by the support device.

The inventors have realised that inconsistency in the volumes of sample dispensed onto lateral flow test devices is a significant source of error in the accuracy of the results reported at mass testing events of the kind discussed above. If too much or too little liquid sample is dispensed onto the device, the analyte and conjugating substance may not react and accumulate in the manner required to produce an accurate visual result as intended. Lateral flow tests of the kind typically used to test for a SARS-COV-2 antigen are particularly vulnerable in this respect since they generally require the operator to dispense, by hand, a predetermined number of drops of sample liquid onto the sample device. While the number of drops can be taken as a rough proxy for the volume of liquid dispensed, the actual volume that arrives on the test device is dependent on the technique of the operator and their judgement in counting the correct number of drops. It is also significantly affected if air bubbles are present in the drops dispensed, which may go unnoticed by the operator, particularly where large numbers of tests are being prepared at speed. By providing a means for dispensing a predetermined volume of sample liquid onto the lateral flow test device(s) in combination with a support device that supports the test devices for this purpose, the present invention eliminates this source of error and thus improves reliability of the results reported. The provision of apparatus configured to dispense liquid onto lateral flow test devices as in the present invention is a counterintuitive step: a major attraction of lateral flow tests, both traditionally and in the context of the coronavirus pandemic, has been that they can be provided as a simple kit with all of the components required to conduct the test (e.g. a swab, buffer solution and capsule for dispensing the sample liquid onto the lateral flow test device) without the need for further specialised equipment. The present invention thus reflects a recognition that the advantages of lateral flow tests in terms of speed, low cost and simplicity of manufacture can be retained while greatly enhancing the quality of the results obtained by providing an apparatus that eliminates operator error in the step of dispensing the liquid sample. The apparatus of the present invention is particularly suitable for mass testing in settings such as airports and event venues, since in these settings the tests will typically be conducted at a fixed checkpoint such as an entryway where the apparatus may be deployed and accessible by all of the operators conducting the tests.

Each of the support devices is a feature of the apparatus that is capable of supporting one or more lateral flow test devices for having liquid samples dispensed onto them. The support device could support the test device(s) simply by providing a surface on which the devices can be placed in use, but could also be adapted to restrain or resiliently hold the device (for example by framing the device on some or all of its sides or by clamping or otherwise urging it into place).

Preferred examples of support devices will be described later. The benefits of the invention are most strongly felt where the one or more support devices are configured to hold (either individually or in combination) a plurality of lateral flow devices, for example more than five. This allows the apparatus to be used to conduct multiple lateral flow tests at the same time. Each individual support device may itself be capable of supporting one or a plurality of lateral flow test devices in use, and the apparatus may comprise one or a plurality of such support devices.

Various examples of dispensing apparatus suitable for dispensing known volumes of liquid samples are known, examples of which will be discussed later. Robotic pipettors are an example of a category of devices suitable for this purpose. In preferred embodiments, the dispensing apparatus is configured to dispense the same volume of liquid sample onto each lateral flow test device in use.

In preferred embodiments, the apparatus further comprises a rotatable carousel adapted to support the, or each, support device in use. The support devices can be placed onto and removed from the carousel in use, and can be loaded with lateral flow test devices before being placed onto the carousel. This is particularly advantageous where the apparatus comprises a chamber inside which the support devices can be placed in use, which will be discussed later. In such a case, the chamber could be provided with one or more openings through which the support devices can be introduced and removed from the chamber, and rotating the part of the carousel onto which the support device is to be placed towards the opening can improve ease of adding the test devices to the carousel for subsequently receiving liquid samples.

As noted above, the apparatus may further comprise a chamber inside which the, or each, support device may be placed in use. This can protect the lateral flow test devices from interference and contamination by debris and the like from the surrounding environment. In particularly preferred embodiments the chamber comprises an opening through which the, or each, support device may be introduced to and removed from the chamber in use. This opening could be provided as a doorway or hatch, for example.

In preferred embodiments, the or each support device is removable from the chamber. The support devices (on which the lateral flow test devices are mounted in use) being removable is advantageous for several reasons. Firstly, it reduces the risk of the lateral flow test devices being contaminated by an operator when they are loaded into the chamber, since the lateral flow test devices do not need to be handled directly once mounted on the support device. Secondly, it improves the ease of loading and unloading lateral flow test devices from the chamber, particularly where each support device is adapted to support a plurality of lateral flow test devices, since the lateral flow test devices can be introduced to the chamber in batches, rather than one at a time. Thirdly, it significantly improves the ease of cleaning and maintaining the parts of the apparatus that are in contact with the lateral flow test devices and onto which the sample is dispensed (i.e. the support devices), since these parts can simply be removed for cleaning. This is advantageous because the support devices are the part of the apparatus that is most liable to contamination by the samples being dispensed onto the lateral flow test devices and hence may require frequent cleaning. This advantage is particularly strongly felt in embodiments in which the support devices comprise the sample receptacles, which will be described later.

Where a chamber is provided, the apparatus preferably further comprises a control system configured to regulate one or more of the temperature, pressure, humidity and light conditions inside the chamber. Variations in each of these factors can affect the development of the tests and how the visual results they produce appear to an operator and/or equipment used to read the results, so this improves the consistency of the tests that are conducted using the apparatus.

Advantageously the, or each, support device may comprise one or more receiving features each adapted to receive a respective one of the lateral flow test devices in use, wherein preferably the or each receiving feature is formed by a recess shaped to cooperate with the perimeter of the received lateral flow test device.

The provision of receiving feature(s) at specific locations on the support device helps to prevent movement of the lateral flow test devices during the dispensing and subsequent development of the test, and helps the dispending apparatus to consistently dispense the liquid samples onto the correct parts of the lateral flow devices. In particularly preferred implementations, each receiving feature is adapted to receive a rectangular lateral flow test device, preferably having lateral dimensions of 69 millimetres (mm)×20 mm. Most lateral flow test devices of the kind used to test for the presence of a SARS-COV-2 antigen are formed as generally flat, rectangular cassettes with lateral dimensions (in this case the dimensions of the rectangular perimeter of the cassette) of 69 mm×20 mm, so these embodiments are particularly suitable for conducting the most widely available SARS-COV-2 lateral flow tests.

Preferably the, or each, sample receptacle is comprised by a respective one of the support devices. In other words, each support device may include one or more sample receptacles. This allows the liquid samples to be loaded into the apparatus together with the lateral flow test devices. As will be described later with reference to particularly preferred embodiments, each sample receptacle could be adapted to receive a sample bottle containing the liquid sample.

Conveniently, the, or each, support device may be formed integrally with the, or each, sample receptacle that it comprises.

The apparatus may preferably further comprise an imaging device arranged to capture images of the lateral flow test devices held by the or each support device inside the chamber in use. This device could be a digital camera, for example. The captured images can be analysed to read the results of the developed lateral flow test devices being handled by the apparatus, either by a human operator reviewing the images (which could be output to a screen or other such display device) or processed in order to computationally determine the results. The imaging device may be configured to capture an image of each lateral flow test device at a predetermined time interval after the respective liquid sample has been dispensed onto said lateral flow test device by the dispending apparatus. This ensures that an image of each test device is captured after the same period of time after the device has received its respective liquid sample, which ensures consistency in the processing of the test devices.

Where the imaging device is provided, it is preferably in communication with a processor configured to process each captured image so as to identify the presence of a predetermined visual result produced by the imaged lateral flow test device and indicative of the presence or absence of an analyte. The term “predetermined visual result” here refers to a visible indicium that is expected to appear on the lateral flow test device when the received sample contains the analyte of interest. For example, a lateral flow test device could be configured such that a visible line forms in a specific region of the device when the analyte is present. A detailed example of this will be described below with reference to an exemplary lateral flow test device. Various techniques may be applied to identify the result of the test in the captured image. For example, an algorithm could be used to identify the presence of a line that is expected to appear if the antigen is present in the sample.

The apparatus preferably further comprises lighting apparatus arranged to substantially uniformly illuminate the, or each, held lateral flow test device. This is particularly advantageous where an imaging device of the kind described above is present since it ensures consistency between images.

The dispensing apparatus may be configured to dispense the same volume of liquid sample onto each of the held lateral flow test devices in use. This is particularly advantageous where lateral flow test devices to be processed by the apparatus are all of the same kind, for example requiring a specified volume such as two drops of sample liquid.

A second aspect of the invention provides a system comprising the apparatus of the first aspect above and one or more lateral flow test devices adapted to be supported by the, or each, support device in use. The, or each, lateral flow test device may be adapted to be supported by the support devices (particularly where the support devices comprise the receiving features described above), for example having a rectangular form, preferably with lateral dimensions of 69×20 mm.

The, or each, lateral flow test device is configured to detect a virus, preferably SARS-COV-2. The, or each, lateral flow test device may be configured to produce a visual result indicative of the presence or absence of an analyte. This could be, for example, the appearance of a coloured test line at a predetermined location on the lateral flow test device.

The system may also comprise one or more sample bottles, wherein the, or each, sample receptacle is adapted to retain a respective one of the sample bottles in use. The provision of sample bottles which can be received by the sample receptacles reduces the risk of cross-contamination between samples as compared to approaches such as manually pouring each sample into a part of the apparatus.

A third aspect of the invention provides a method of conducting one or more lateral flow tests, the method comprising: providing the apparatus of the first aspect of the invention or the system of the second aspect of the invention; placing one or more lateral flow test devices onto the support device so as to be supported thereby, each of the lateral flow test devices comprising a sample area and a test area in fluid communication with the sample area; depositing each of one or more liquid samples into the one or more sample receptacles; and dispensing, by the dispensing apparatus, the same predetermined volume of each of the liquid samples onto the sample area of a respective one of the lateral flow test devices held by the support device.

The method preferably further comprises reading a result of the, or each, lateral flow test device at a predetermined time after the respective liquid sample was dispensed onto that lateral flow test device. This can be done manually by an operator. However, in preferred embodiments, reading the result comprises capturing an image of the lateral flow test device and processing the image to identify a predetermined visual result indicative of the presence or absence of an analyte. The capturing and processing of the image could be performed by the imaging device and processor described previously with respect to preferred embodiments of the first aspect.

### BRIEF DESCRIPTION OF THE DRAWINGS

Examples of apparatus, systems and methods in accordance with the invention will now be described with reference to the accompanying drawings, in which:

FIGS. 1(a) and 1(b) show an example of a lateral flow test device suitable for use with apparatus in accordance with embodiments of the invention;

FIG. 2 shows an example of an apparatus in accordance with an embodiment of the first aspect of the invention;

FIGS. 3(a), 3(b) and 3(c) are detailed views of a carousel and supporting devices suitable for use in preferred embodiments of the invention; and

FIGS. 4(a), 4(b), 4(c) and 4(d) show additional features that are incorporated in preferred embodiments of the apparatus of FIG. 2.

### DETAILED DESCRIPTION

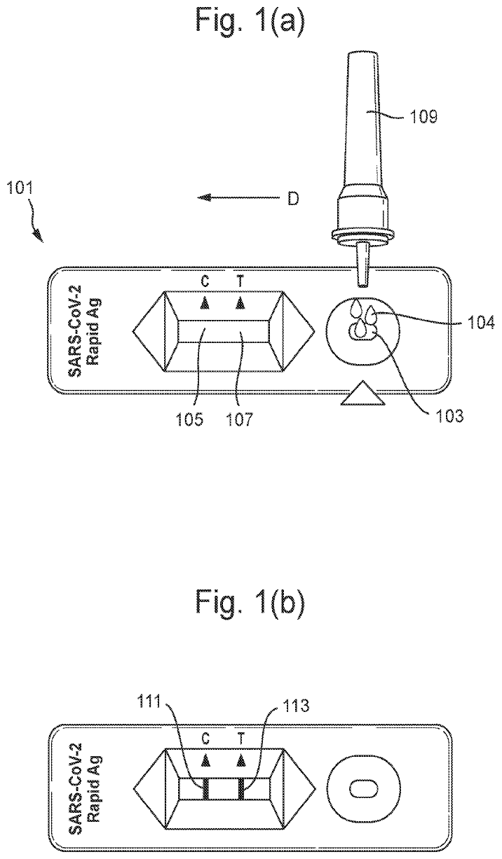

FIG. 1(a) shows in plan view an example of a lateral flow test device 101, which may be suitable for use with apparatus in accordance with embodiments of the invention. The lateral flow test device 101 has the form of a generally rectangular cassette which encloses a strip of porous material such as paper or a microstructured polymer. On the side visible in FIG. 1(a), the cassette has openings that expose parts of the strip of porous material for depositing a liquid sample and reading the result of the lateral flow test. Part of the strip comprises a sample pad 103, which is exposed by one of the openings. In order to perform a lateral flow test using this device 101, drops of a liquid sample 104 are dispensed onto the sample pad 103, which quickly absorbs the liquid. Conventionally the liquid sample 104 has been dispensed using manual apparatus such as a dispensing tube 109, which is squeezed by the operator in order to eject liquid onto the sample pad 103. After being dispensed onto the sample pad 103, the liquid 104 diffuses along the porous strip in the direction D labelled in FIG. 1(a). Spaced from the sample pad 103 along the D direction (in a region of the strip not exposed by the cassette) is a quantity of conjugating substance, which contains a visual identifier such as colloidal gold and mixes with the liquid sample as it diffuses along the strip. The conjugating substance is configured to bind to or react with an analyte of interest (for example a virus, or fragments thereof) the presence of which the device 101 is intended to test for. If the analyte of interest is present, the conjugating substance binds to or reacts with the analyte and the resulting conjugated analyte travels further along the strip in the D direction. Yet further along the strip in the D direction is a test zone 107. The test zone 107 contains a substance such as antibodies that are configured to trap the conjugated analyte. If the analyte is present, this will cause the conjugated analyte to concentrate in the test zone 107, which will eventually result in a visible colour change in that zone. FIG. 1(b) shows the device 101 of FIG. 1(a) showing a positive result, which can be identified by the presence of a visible test line 113 in the test zone 107. Many lateral flow test devices also include a control zone 105 spaced along the strip from the test zone 107. The control zone is configured to trap non-conjugated molecules of the conjugating substance. Since not all of the conjugating substance will encounter the analyte even where the analyte is present, a visible control line 111 should therefore develop whether or not the analyte is present. The appearance of a control line 111 after the test has been allowed to develop therefore verifies that the conjugating substance was indeed present in the strip at the outset and that it has been able to travel the length of the strip. Typically the lateral flow test device 101 is inspected at a predetermined time after the liquid sample has been dispensed onto it (for example 30 minutes) and a positive or negative result is declared depending on whether a test line 113 is visible at that time.

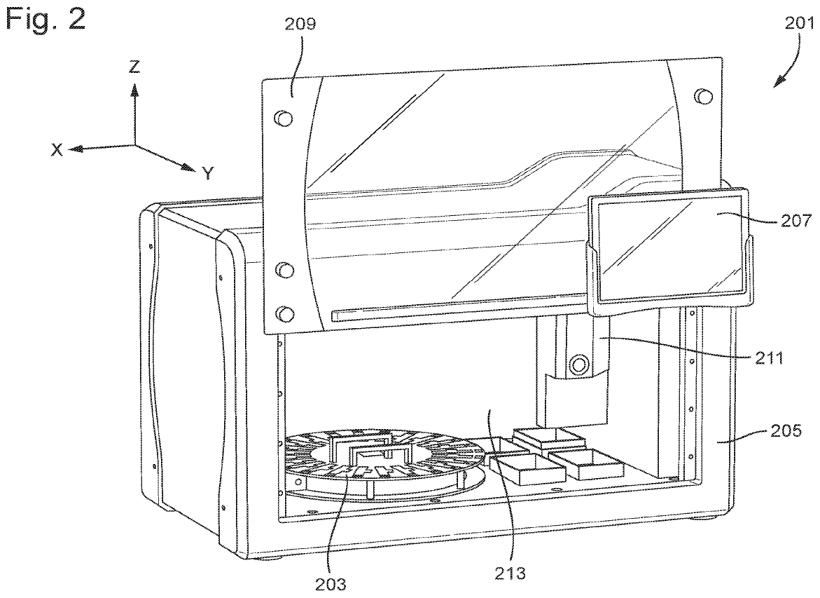

FIG. 2 shows an example of an apparatus 201 in accordance with an embodiment of the invention. The apparatus 201 includes a chamber 205, the interior of which is accessible through an opening that is closed in use by a transparent sliding door 209. A rear wall 213 of the chamber is formed by a transparent screen through which lateral flow test devices inside the chamber in use may be inspected. The transparent parts of the chamber 205 (in the case sliding door 209 and rear wall 213) may be tinted in order to reduce the quantity of ambient light inside the chamber, which improves the consistency of the conditions under which the lateral flow test devices therein are inspected to identify the results of the tests conducted (whether by visual inspection or capturing images of the devices). The apparatus 201 may include apparatus configured to regulate environmental conditions such as the temperature, humidity and light inside the chamber 205

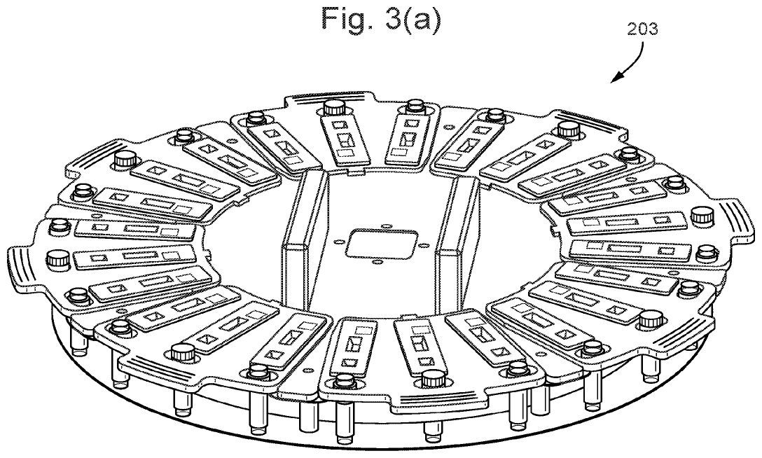

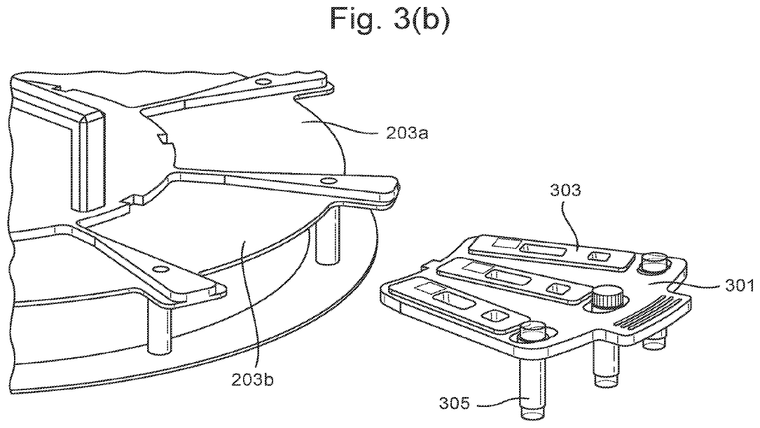

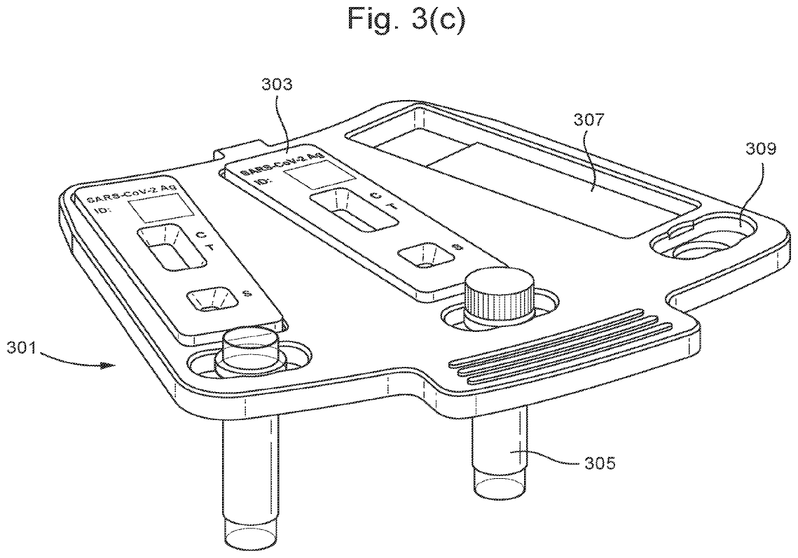

Inside the chamber 205 is a carousel 203, which carries a plurality of support devices (which will be described later with reference to FIG. 3) each adapted to support a plurality of lateral flow test devices in use. In this embodiment, the support devices carried by the carousel are each adapted to support a plurality of lateral flow test devices 101 of the kind described above with reference to FIGS. 1(a) and 1(b). The support devices carried by the carousel also comprise sample receptacles adapted to receive liquid samples contained in sample bottles. In use, the carousel is loaded with a plurality of lateral flow test devices and corresponding liquid samples each to be dispensed onto a respective one of the lateral flow test devices. The carousel 203 is rotatable about its centre in the plane perpendicular to the Z axis, which improves ease of loading and unloading test devices from it. The rotation of the carousel in this example is driven by a motor, which may be controlled by a processor in order to manage the positions of the lateral flow test devices and samples supported on it in use. The support devices are removable from the carousel 203 and the chamber 205.

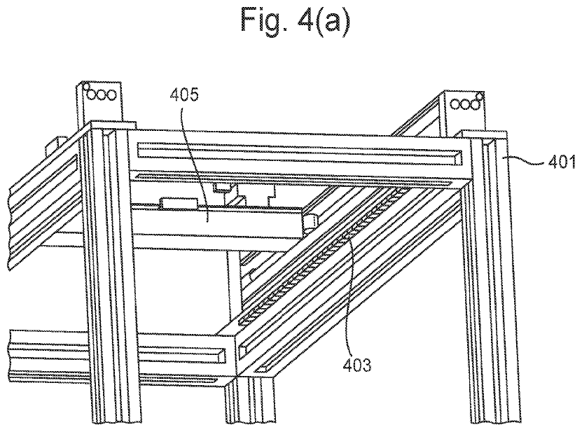

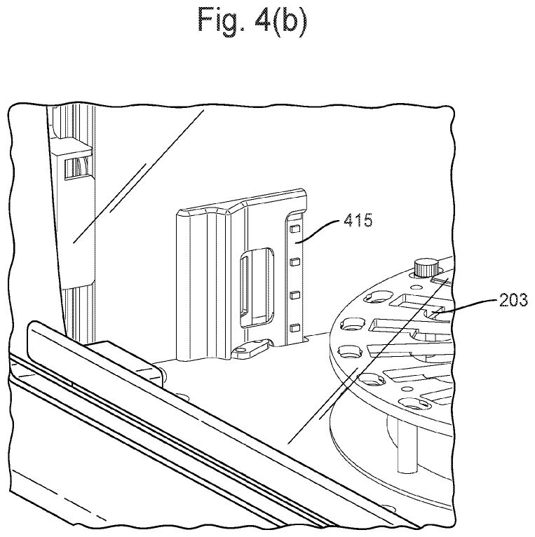

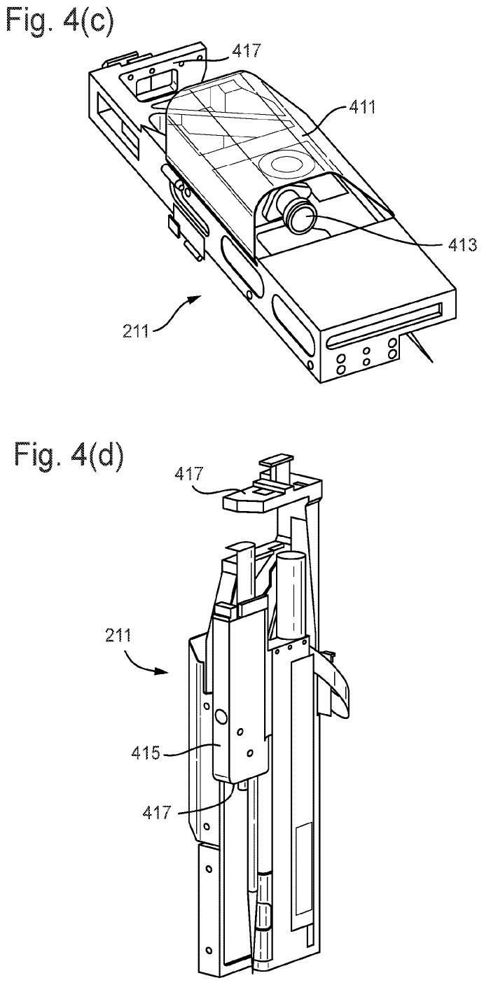

The apparatus 201 includes a dispensing apparatus (which will be described in detail later with reference to FIG. 4(d)) that includes a robotic pipettor, which aspirates the liquid samples from the received sample bottles and dispenses the same predetermined volume of each liquid sample onto its respective lateral flow test device. Once a liquid sample has been dispensed onto a lateral flow test device, it begins to develop in the manner described above. In this embodiment, the dispensing apparatus is provided as part of a moveable assembly 211 that also includes an imaging device (which will be described later with reference to FIG. 4(c)) and which can be moved along the X and Y directions. The imaging device is configured to capture an image of each lateral flow test device at a predetermined time interval after the dispensing apparatus has dispensed the respective liquid sample onto it. The captured image is received by a processor (for example one incorporated in the apparatus 201 or provided by a separate computer in communication with the apparatus 201), which processes the image in order to identify whether a test line is visible in the part of the image corresponding to the test zone 107 of the lateral flow test device 101. The identification of the test line may be performed by applying an image processing algorithm configured to identify the predetermined visual result (such as a test line) in the relevant portion of the image, for example.

FIGS. 3(a) to 3(c) show detailed views of parts of the carousel 203. As can be best seen in FIG. 3(b), the carousel is divided into a plurality of segments 203a, 203b each of which is shaped to receive a support device 301 to be supported on the carousel in use. Each support device 301 has a generally planar form and comprises a plurality of receiving features 307 (in this example three) formed by recesses in the device. Each receiving feature 307 is shaped to receive a lateral flow test device 303 (such as that described previously with reference to FIGS. 1(a) and 1(b)). Adjacent to each receiving feature is a sample receptacle 309, which formed by an opening in the support device 301 that is adapted to receive and support a sample bottle 305 in use. In alternative embodiments, the sample receptacle could be formed as a well or recess in the support device 301 configured to receive the liquid sample directly (not in a separate container such as a bottle), but the use of sample bottles is preferred because this reduces the risk of contamination of the apparatus by the liquid samples and avoids the need to clean the support devices between uses. In use, an operator will place unused lateral flow test devices 303 into the recesses of the support device and put sample bottles 305 containing liquid samples (prepared for example by mixing a swab sample with buffer solution inside the sample bottle) into the corresponding sample receptacles 309. The support device 301 is then placed onto the carousel 203 inside the chamber 205. The dispensing apparatus inside the apparatus 201 can then proceed to aspirate the liquid samples from the sample bottles 305 and dispense them onto the corresponding lateral flow test devices 303 in order to commence the assay in the manner described previously.

FIGS. 4(a) to 4(c) show additional details of features that are present inside the apparatus in this embodiments. FIG. 4(a) shows part of a framework 401 which is positioned over the carousel 203 inside the chamber 205. The framework 401 support a sliding carriage 405, which carries the assembly 211 that includes the imaging device and dispensing apparatus and is movable (for example by a motor controlled by a processor) to position the imaging device over the different lateral flow test devices supported by the carousel 203 in use. Lights 403, in this example in the form of LED strips, are arranged on the framework to illuminate the carousel in use. Although only one set of lights 403 is visible in this drawing, there is preferably a similar set of lights arranged on the part of the framework 401 opposite the lights 403 shown, which further improves the uniformity of illumination. The provision of lights 403 improves the consistency of the images captured by the imaging device and in particular can prevent the appearance of shadows in the capture images, which may be misread as positive test results by a processor configured to identify test results in the captured images. A further light 415 (again an LED strip in this example) may be arranged laterally adjacent to the carousel 203 at the base of the chamber 205 as shown in FIG. 4(b). This is particularly advantageous where the lateral flow test devices carry identifying indicia such as barcodes, which may be read by the imaging device and recorded by the processor in order to lateral associate each test result with the sample on which the test was conducted.

FIG. 4(c) shows a detailed view of the part of the assembly 211 that carries the imaging device, which in this example is a camera 413. The camera 413 is positioned so as to face downwards towards the carousel 203 when the assembly 211 is mounted on the sliding carriage 405 in use. A tinted cover 411 is arranged over the camera 413. This helps to prevent the captured images being affected by glare, which may be caused by ambient light sources (e.g. sunlight or artificial lighting in the surrounding environment) and also from the lights 413, 415 inside the chamber 205.

5 FIG. 4(d) shows the reverse side of the assembly 211, which carries the dispensing apparatus. The dispensing apparatus in this example is a pipettor 415, which has an attachment point 417 to which a disposable pipette tip can be fixed in use. A liquid sample will be aspirated by the pipettor 415 into the attached pipette tip and then dispensed onto the corresponding lateral flow test device 303 from the same pipette tip. Each pipette tip will typically be used to handle a single sample and will be removed from the pipettor 415 (either for disposal or for cleaning and re-use) after dispensing the liquid sample. The use of separate pipette tips for each sample prevents cross-contamination between different samples and hence improves the accuracy of the results recorded.

1. An apparatus for assaying one or more lateral flow test devices, the apparatus comprising:
at least one support device for supporting one or more lateral flow test devices in use;
at least one sample receptacle configured to receive, in use, a respective liquid sample to be dispensed onto a corresponding lateral flow test device; and
a dispensing apparatus configured to dispense a known volume of the received liquid sample from the sample receptacle onto the corresponding lateral flow test device supported by the support device.

2. The apparatus of claim 1, further comprising a rotatable carousel adapted to support the or each support device in use.

3. The apparatus of claim 1, further comprising a chamber inside which the, or each, support device may be placed in use; wherein preferably the, or each, support device is removable from the chamber.

4. (canceled)

5. The apparatus of claim 3, wherein the chamber comprises an opening through which the, or each, support device may be introduced to and removed from the chamber in use.

6. The apparatus of claim 3, further comprising a control system configured to regulate one or more of the temperature, pressure, humidity and light conditions inside the chamber.

7. The apparatus of claim 1, wherein the, or each, support device comprises one or more receiving features each adapted to receive a respective one of the lateral flow test devices in use, wherein preferably the, or each, receiving feature is formed by a recess shaped to cooperate with the perimeter of the received lateral flow test device.

8. The apparatus of claim 7, wherein each receiving feature is adapted to receive a rectangular lateral flow test device, preferably having lateral dimensions of 69 mm×20 mm.

9. The apparatus of claim 1, wherein the, or each, sample receptacle is comprised by a respective one of the support devices; wherein preferably each support device is formed integrally with the, or each, sample receptacle that it comprises.

10. (canceled)

11. The apparatus of claim 1, further comprising an imaging device arranged to capture images of the lateral flow test devices held by the, or each, support device inside the chamber in use.

12. The apparatus of claim 11, wherein the imaging device is configured to capture an image of each lateral flow test device at a predetermined time interval after the respective liquid sample has been dispensed onto said lateral flow test device by the dispending apparatus.

13. The apparatus of claim 12, wherein the imaging device is in communication with a processor configured to process each captured image so as to identify the presence of a predetermined visual result produced by the imaged lateral flow test device and indicative of the presence or absence of an analyte.

14. The apparatus of claim 1, further comprising lighting apparatus arranged to substantially uniformly illuminate the, or each, held lateral flow test device.

15. The apparatus of claim 1, wherein the dispensing apparatus is configured to dispense the same volume of liquid sample onto each of the held lateral flow test devices in use.

16. A system comprising the apparatus of claim 1 and one or more lateral flow test devices adapted to be supported by the, or each, support device in use; wherein preferably the, or each, lateral flow test device has a rectangular form, preferably with lateral dimensions of 69×20 mm.

17. (canceled)

18. The system of claim 16 wherein the, or each, lateral flow test device is configured to detect a virus, preferably the SARS-COV-2 antigen.

19. The system of claim 16, wherein the, or each, lateral flow test device is configured to produce a visual result indicative of the presence or absence of an analyte.

20. The system of claim 16, further comprising one or more sample bottles, wherein the, or each, sample receptacle is adapted to retain a respective one of the sample bottles in use.

21. A method of conducting one or more lateral flow tests, the method comprising:
providing the apparatus or system of claim 1;
placing one or more lateral flow test devices onto the support device so as to be supported thereby, each of the lateral flow test devices comprising a sample area and a test area in fluid communication with the sample area;
depositing each of one or more liquid samples into the one or more sample receptacles; and
dispensing, by the dispensing apparatus, the same predetermined volume of each of the liquid samples onto the sample area of a respective one of the lateral flow test devices held by the support device.

22. The method of claim 21, further comprising reading a result of the, or each, lateral flow test device at a predetermined time after the respective liquid sample was dispensed onto that lateral flow test device.

23. The method of claim 22, wherein reading the result comprises capturing an image of the lateral flow test device and processing the image to identify a predetermined visual result indicative of the presence or absence of an analyte.
